(19) United States
(12) Patent Application Publication
(10) Pub. No.: US 2025/0321204 A1
(43) Pub. Date: Oct. 16, 2025

(54) SENSOR UNIT OF A NUCLEIC ACID ANALYSIS SYSTEM

(71) Applicant: Robert Bosch GmbH, Stuttgart (DE)

(72) Inventors: Martina HUEBNER, Renningen (DE); Daniel PANTEL, Mundelsheim (DE); Jonas OTT, Boeblingen (DE); Robert SCHARF, Garbsen (DE); Franz LAERMER, Weil Der Stadt (DE); Christopher JOHNSON, San Carlos, CA (US); Gabrielle VUKASIN, San Jose, CA (US); Armin DARVISH, Brisbane, CA (US); Gary YAMA, Mountain View, CA (US); Young Shik SHIN, Mountain View, CA (US)

(21) Appl. No.: 18/635,090

(22) Filed: Apr. 15, 2024

(51) Int. Cl. G01N 27/327; B82Y 15/00; C12Q 1/6869

(52) U.S. Cl. CPC G01N 27/3278; C12Q 1/6869; B82Y 15/00

(57) ABSTRACT

A method for producing a sensor unit for nucleic acid analysis having a first electrode and a second electrode in a nanochannel, the method includes arranging a main direction of extension of the nanochannel at an angle γ from about 70° to about 110° to a direction of extension of edges of exposed surface portions of the first and second electrodes.

### TECHNICAL FIELD

The present disclosure relates to a sensor unit for nucleic acid analysis, in particular DNA and/or RNA analysis, a system including the sensor unit, and a method of producing the same.

### BACKGROUND

In a method for analyzing DNA, modified DNA strands are passed past a pair of electrodes that are spaced in the nanometer range and a redox potential is applied to the pair of electrodes so that a current flow is measured. This current flow depends on both the redox potential applied to the electrode pair spaced in the nanometer range and the precisely modified nucleotide of the DNA strand, which is located in the detection range of the electrode pair.

In WO 2015/111760 A1, EP 3 109 627 A1, and EP 3 704 438 B1, respectively sensor units for DNA analysis with electrodes that are spaced from one another in the nanometer range are disclosed.

### SUMMARY

In one embodiment, a method for producing a sensor unit for nucleic acid analysis having a first electrode and a second electrode in a nanochannel is disclosed. The method may include arranging a main direction of extension of the nanochannel at an angle γ from about 70° to about 110° to a direction of extension of edges of exposed surface portions of the first and second electrodes. The angle γ may be about 90°. The method may further include depositing a first electrode material to form the first electrode in step (S100). The method may further include depositing an intermediate layer material to form an intermediate layer between the first and second electrodes such that the intermediate layer defines an electrode distance between the first and second electrodes in step (S200). The method may further include depositing a second electrode material on the intermediate layer to form the second electrode in step (S300). The method may further include structuring the first electrode material of the first electrode, an intermediate layer material of the intermediate layer, the second electrode material of the second electrode, or their combination to form a chamfer with at least one chamfer angle α in step (S400). The method may further include depositing a sacrificial layer material on the first electrode, the intermediate layer, the second electrode, or their combination to form a sacrificial layer, and depositing a coating material on the sacrificial layer to form a coating layer in step (S500). The method may further include structuring the sacrificial layer and/or the coating material in step (S600). The method may further include partially under-etching the deposited and structured sacrificial layer to form the nano-channel in step (S700). The method may further include depositing an additional coating material on the deposited coating material to form an additional coating layer to close the nanochannel in step (S800).

In another embodiment, a nucleic acid analysis sensor unit is disclosed. The unit may include a first and second electrodes located in a nanochannel, the first and second electrodes each having exposed surface portions, the nanochannel having a main direction of extension arranged at an angle γ from about 70° to about 110° to a direction of extension of edges of the exposed surface portions. The angle γ may be about 90°. The unit may also include an intermediate layer material forming at least one intermediate layer between the first and second electrodes, the intermediate layer defining an electrode distance between the first and second electrodes. The intermediate layer may have a thickness of about 1 nm to about 10 nm. The electrode distance may be about 1 nm to about 5 nm. The first electrode, the intermediate layer, the second electrode, or a combination thereof may include a chamfer with at least one chamfer angle α. The at least one chamfer angle α may be in a range from about 0° to about 85°. The at least one chamfer angle α may be in a range from about 30° to about 70°. The unit may also include a structured sacrificial layer material on the first electrode, the intermediate layer, the second electrode, or a combination thereof. The unit may also include a first coating layer configured on the structured sacrificial layer. The unit may also include a second coating material on the first coating material, the second coating material closing the nanochannel.

In yet another embodiment, a DNA or RNA sequencing analysis system is disclosed. The system may include first and second containers connected by a nanochannel; a nucleic acid analysis sensor unit including a first and second electrodes, located in the nanochannel, adjacent each other and each including one or more exposed surface portions with one or more edges. The nanochannel may have a main direction of extension arranged at an angle γ from about 70° to about 110° to a direction of extension of the one or more edges. The unit may further include an intermediate layer located between and separating the first and second electrodes, the separation measuring about 1 nm to about 5 nm. The first electrode, the intermediate layer, the second electrode, or a combination thereof may include a chamfer with at least one chamfer angle α of about 0° to about 85°. The unit may further include a structured sacrificial layer material on the first electrode, the intermediate layer, the second electrode, or a combination thereof. The unit may further include a first coating layer configured on the structured sacrificial layer.

### BRIEF DESCRIPTION OF THE DRAWINGS

FIG. 1 is a schematic representation of non-limiting example components of an analysis unit for nucleic acid analysis, in particular for DNA analysis and/or for RNA analysis, according to one or more embodiments disclosed herein;

FIG. 2 is a top view of the unit shown in FIG. 1 with the lower layer structures being partially exposed;

FIG. 3 is a schematic representation of a sectional view along section line I-I of the unit of FIG. 2; and

FIGS. 4-11 are schematic representations of manufacturing steps for producing the unit shown in FIGS. 2 and 3 along the section line II-II in FIG. 2.

### DETAILED DESCRIPTION

Embodiments of the present disclosure are described herein. It is to be understood, however, that the disclosed embodiments are merely examples and other embodiments may take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the present embodiments. As those of ordinary skill in the art will understand, various features illustrated and described with reference to any one of the figures may be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Except in the examples, or where otherwise expressly indicated, all numerical quantities in this description indicating amounts of material or conditions of reaction and/or use are to be understood as modified by the word “about” in describing the broadest scope of the disclosure. Practice within the numerical limits stated is generally preferred. Also, unless expressly stated to the contrary: percent, “parts of,” and ratio values are by weight; the description of a group or class of materials as suitable or preferred for a given purpose in connection with the disclosure implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed. Unless stated otherwise, the wt. % is based on the total weight of the substrate and the vol. % is based on the total volume of the substrate.

The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “substantially,” “generally,” or “about” means that the amount or value in question may be the specific value designated or some other value in its neighborhood. Generally, the term “about” denoting a certain value is intended to denote a range within +/−5% of the value. As one example, the phrase “about 100” denotes a range of 100+/−5, i.e. the range from 95 to 105. Generally, when the term “about” is used, it can be expected that similar results or effects according to the disclosure can be obtained within a range of +/−5% of the indicated value. The term “substantially” may modify a value or relative characteristic disclosed or claimed in the present disclosure. In such instances, “substantially” may signify that the value or relative characteristic it modifies is within +0%, 0.1%, 0.5%, 1%, 2%, 3%, 4%, 5% or 10% of the value or relative characteristic.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4, . . . , 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. Similarly, whenever listing integers are provided herein, it should also be appreciated that the listing of integers explicitly includes ranges of any two integers within the listing.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

As used herein, the term “and/or” means that either all or only one of the elements of said group may be present. For example, “A and/or B” means “only A, or only B, or both A and B”. In the case of “only A,” the term also covers the possibility that B is absent, i.e. “only A, but not B”.

It is also to be understood that this disclosure is not limited to the specific embodiments and methods described below, as specific components and/or conditions may, of course, vary. Furthermore, the terminology used herein is used only for the purpose of describing particular embodiments of the present disclosure and is not intended to be limiting in any way.

The term “comprising” is synonymous with “including,” “having,” “containing,” or “characterized by.” These terms are inclusive and open-ended and do not exclude additional, unrecited elements or method steps. The term “including” or “includes” may encompass the phrases “comprise,” “consist of,” or “essentially consist of.”

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed subject matter can include the use of either of the other two terms.

The term “one or more” means “at least one” and the term “at least one” means “one or more.” The terms “one or more” and “at least one” include “plurality” as a subset.

The description of a group or class of materials as suitable for a given purpose in connection with one or more embodiments implies that mixtures of any two or more of the members of the group or class are suitable. Also, the description of a group or class of materials as suitable for a given purpose in connection with one or more embodiments implies that the group or class of materials can “comprise,” “consist of,” and/or “consist essentially of” any member or the entirety of that group or class of materials. First definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

The term nucleic acid analysis is used, for example, to describe the sequencing of DNA and/or RNA, in other words the determination of the nucleotide sequence in a DNA and/or RNA molecule. The analysis may be conducted in a system or unit including a sensor unit disclosed herein.

In one or more embodiments, a method for producing a sensor unit having two electrodes in a nanochannel for nucleic acid analysis is disclosed. The analysis may be DNA analysis and/or for RNA analysis. The main direction of extension of the nanochannel is arranged such that it extends at an angle (γ) from about 70° to about 110° to a direction of extension of the sensor unit—more specifically to a direction of extension of edges of exposed surface portions of a first electrode and a second electrode of the sensor unit. The angle may be about 70-110, 75-100, or 85-95°. The angle may be about, at least about, or at most about 70, 71, 72, 73, 74, 75, 76, 77, 78, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110°.

This type of arrangement is particularly advantageous because it enables the nucleic acids to be analyzed precisely while being guided through the detection area of the sensor unit or the nano-gap in the nanochannel.

When the main direction of extension of the nanochannel extends at an angle (γ) from about 70° to about 110° to a direction of extension of the edges of the exposed surface portions of the first electrode and the second electrode, a particularly advantageous detection is possible due to the small effective distance between the electrodes in the nano-channel. Particularly advantageous is an angle (γ) of about 90°, as this is the smallest effective distance between the electrodes in the nano-channel.

In one or more embodiments, the method may include a deposition of a first electrode material to form the first electrode. The method may include a deposition of an intermediate layer material to form an intermediate layer between the first and second electrode. The intermediate layer defines, for example, an electrode distance between the two electrodes or—if there are several layers between the electrodes—contributes to determining the same. The method may include a deposition of a second electrode material on the intermediate layer to form the second electrode.

A nanochannel refers to a channel for transporting fluids with a width and height of respectively less than about 100 nm, less than 10 nm, preferably less than 3 nm. The width, height, or their combination may be about or at most about 100, 90, 80, 70, 60, 50, 40, 30, 20, 10, 9, 8, 7, 6, 5, 4, or 3 nm. The first and second electrodes are arranged one above the other above a substrate lying transverse to it. The two electrodes are thus not arranged on two opposite walls of the nanochannel and consequently do not have a distance that is determined by the width of the nanochannel. Rather, the distance between the two electrodes is completely independent of the width of the nano-channel. In particular, the distance between the two electrodes is not limited by a minimum resolvable structure size or resolution limit of a lithography but is instead determined by a freely selectable layer thickness.

In other words, the electrode distance may be defined by the thickness of an intermediate layer, which may be, in a non-limiting example, at least about 1 nm. The thickness of the intermediate layer may be limited downwards solely by a required minimum thickness in the deposition of the intermediate layer material (with regard to process control and reproducibility of the process), the required electrical voltage and breakdown strength (electrical voltage strength of the layer material, insulating properties and pinhole density behavior depending on the layer thickness), or both. Therefore, an electrode distance with a size well below the resolvable structure sizes of a lithography of about 4 nm can be achieved. The electrode distance, for example, is only downwardly limited by the details of the deposition process or the required electrical insulation properties or electrical voltage strengths. This allows the performance of nucleic acid analysis, in particular DNA analysis such as a DNA sequencing or RNA analysis such as an RNA sequencing.

According to one or more embodiments, in a further step, the method may include structuring or “etching through” of a chamfer. The term “structuring” in this disclosure refers to creating, shaping by adding, changing, or removing material. Etching refers to structuring during which existing material is physically and/or chemically removed. The method may include structuring the first electrode material used for a first electrode, the intermediate layer material used for the intermediate layer, the second electrode material used for the second electrode to form a chamfer on the first electrode, second electrode, or both.

A chamfer may be a beveled surface portion, for example in relation to a surface of the substrate, where the two electrodes with the intermediate layer have been deposited successively. The chamfer is formed, for example, by an anisotropic etching process such as by ion beam etching (IBE) or reactive ion etching (RIE). The advantage of the chamfer includes relatively easy application of one or more coatings without undesirable formation of cavities or fissures.

In a non-limiting example, chamfer angles may be set in the range from about 40° to about 50°, about 42° to about 48°, or about 44° to about 46°, making it particularly advantageous for conformal covering.

According to one or more embodiments, the method may include the following one or more subsequent steps:
depositing a sacrificial layer material on the first electrode material (before or after structuring), on the intermediate layer material (before or after structuring), on the second electrode material (before or after structuring), or their combination, to form a sacrificial layer,
depositing a coating material on the sacrificial layer material to form a coating layer,
structuring the sacrificial layer material, the coating material, or both, and
partially under-etching of the deposited and/or structured sacrificial layer material to form the nano-channel.

The partial under-etching to form the nanochannel may be conducted by removing the structured sacrificial layer material. The partial under-etching may be conducted with a ratio of approximately 1 to 1, in relation to the thickness of the structured sacrificial layer material to the lateral under-etching depth, running parallel to the surface of the substrate. For partial under-etching, in a non-limiting example, aqueous tetramethylammonium hydroxide solution (TMAH) or xenon difluoride (XeF₂) may be used as a gas.

According to a further embodiment, the method may include a subsequent step of depositing an additional coating material on the deposited coating material to form an additional coating layer to close the nano-channel.

By depositing the additional coating material with sufficient edge coating, the nanochannel formed by the under-etching is then closed. In a non-limiting example, the closing of the nanochannel may be conducted along one side in the direction of its longitudinal extension. It may be advantageous to use a gaseous etching medium such as, for example, xenon difluoride (XeF₂). Other materials and processes are contemplated.

A sensor unit for nucleic acid analysis is disclosed herein. The unit has two electrodes in a nanochannel. The nanochannel is arranged to have the main direction of extension at an angle (γ) from 70° to 110° to a direction of extension of the edges of the exposed (free) surface portions of the first electrode and the second electrode. The angle may be about 90°. The angle may be about 70-110, 75-100, or 85-95°. The angle may be about, at least about, or at most about 70, 71, 72, 73, 74, 75, 76, 77, 78, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, or 110°. The resulting advantages have been described above.

According to one or more embodiments, an intermediate layer, which contributes to determining, in particular defining, an electrode distance between the first and second electrodes, is arranged between the two electrodes. In one or more embodiments, the intermediate layer may have a thickness in the range from about 1 nm to about 10 nm such as from about 1 nm to about 5 nm, or from about 2.5 nm to about 3.5 nm. The thickness may be about, at least about, or at most about 1, 1.5, 2, 2.5, 3, 3.5, 4, 4.5, 5, 5.5, 6, 6.5, 7, 7.5, 8, 8.5, 9, 9.5, or 10 nm.

As was discussed above, the first electrode material used for the first electrode, the intermediate layer material used for the intermediate layer, the second electrode material used for the second electrode, or their combination may be structured to form a chamfer. The chamfer has at least one chamfer angle (α).

The at least one chamfer angle (α) may be in a range from about 0° to about 85°, from about 30° to about 70°, or from about 40° to about 50°. The at least one chamfer angle (α) may be about, at least about, or at most about 0, 5, 10, 15, 20, 25, 30, 35, 40, 45, 50, 55, 60, 65, 70, 75, 80, or 85°.

In one or more embodiments, a deposited and structured sacrificial layer material may be incorporated on the first electrode (before or after structuring such that the first electrode is structured or unstructured), on the intermediate layer material (before or after structuring such that the intermediate layer is structured or unstructured), on the second electrode (before or after structuring such that the second electrode is structured or unstructured), or their combination to form a sacrificial layer. A deposited and structured coating material may be incorporated on the sacrificial layer material to form a coating layer. A nanochannel formed by at least partial under-etching of the deposited and structured sacrificial layer material may also be incorporated.

Furthermore, an additional deposited coating material may be arranged on the coating material. The additional deposited coating material thus forms an additional coating layer to close the nanochannel.

Moreover, an analysis system/unit including the herein-disclosed sensor unit for nucleic acid analysis is disclosed. The system, sensor unit, or both may be for DNA sequencing and/or for RNA sequencing.

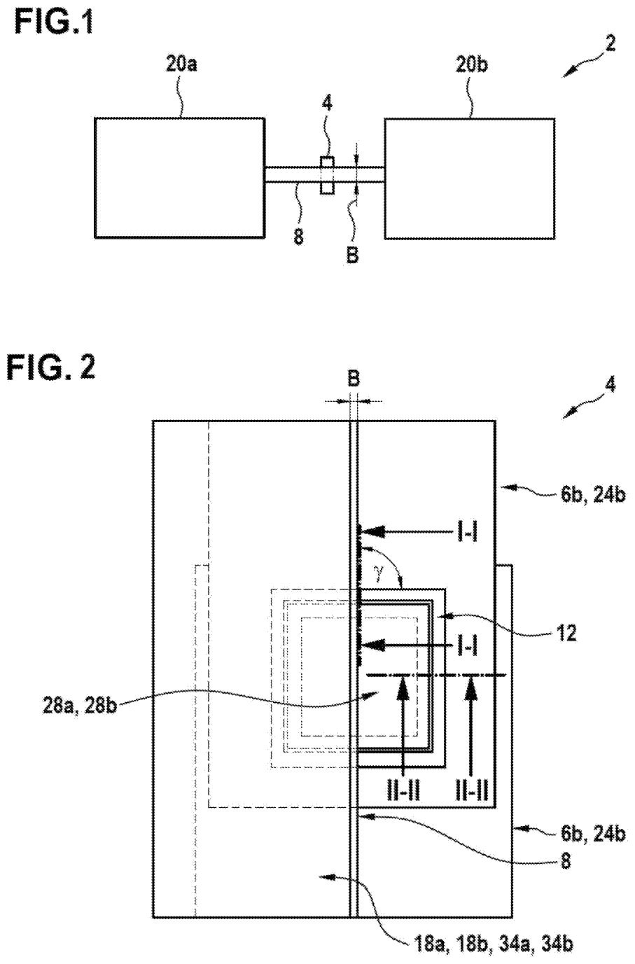

In FIG. 1, example components of an analysis system/unit (2) for nucleic acid analysis, in particular for DNA analysis such as DNA sequencing and/or for RNA analysis such as RNA sequencing, are shown. In the shown components of the analysis system (2), there is a sensor unit (4) having at least two electrodes (6a, 6b) (not shown in FIG. 1) in a nanochannel (8). The nanochannel (8) connects a first container (20a) of the unit (2) to a second container (20b) of the unit (2) while conveying a medium.

In operation of the analysis unit (2), for example, DNA strands modified for DNA analysis are transferred from the first container (20a) to the second container (20b) through the nanochannel (8). The transfer may be conducted, for example, by a pump (not shown) or by an electric field that is applied to the nanochannel (8), or by a concentration gradient by diffusion from the first container (20a) to the second container (20b). The modified DNA strands pass the at least two electrodes (6a, 6b), which are used with a measuring apparatus (not shown) to measure a current flow based on the applied redox potential between the two electrodes (6a, 6b). The current flow is characteristic of the respective modified nucleotide.

In this case, for example, the DNA and/or RNA molecules are modified with redox-active groups, wherein, for example, two bases of the DNA (C, T, A, G) and/or the RNA (C, U, A, G) are provided with a specific redox potential. Alternatively, three of the bases or all four bases can be modified with redox-active groups, wherein every modified base attains a specific redox potential.

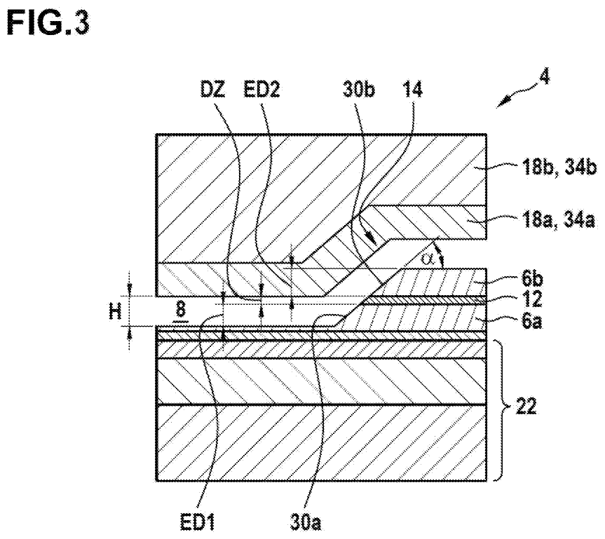

FIGS. 2 and 3 show respectively a top view of the sensor unit (4) with partially exposed lower layer structures (FIG. 2) and a sectional view of the sensor unit (4) (FIG. 3) along the sectional line I-I in FIG. 2.

In a non-limiting example of the FIGS. 2 and 3, the sensor unit (4) is configured as a MEMS component (MEMS—micro-electromechanical system). In a non-limiting example, the MEMS may be manufactured to be CMOS compatible (CMOS—complementary metal-oxide-semiconductor).

The sensor unit (4) may have a substrate (22) (see FIG. 3) such as a silicon wafer, which may have one or more or a plurality of layers, for example a doped layer, such as an epitaxial layer, an aluminum oxide (Al₂O₃) layer, an adhesion promoter layer, or their combination.

The first electrode (6a) of the two electrodes (6a, 6b) is formed on the substrate (22). An intermediate layer (12) is formed on the first electrode (6a). The second electrode (6b) is formed on the intermediate layer (12). When combined, the first electrode (6a), the intermediate layer (12), and the second electrode (6b) form a so-called nanogap.

In a non-limiting example, the first electrode (6a) and/or the second electrode (6b) are made of platinum (Pt) or titanium nitride (TiN). Alternatively, the first electrode (6a) and/or the second electrode (6b) may be made of a different material that is electrically conductive and/or electrochemically active such as gold (Au) or silver (Ag).

In a non-limiting example, the first electrode (6a) is structured on the substrate (22). The intermediate layer (12) may have a first recess (28a) which exposes the first electrode (6a). The recess (28a) may have square shape or another shape. Thus, the first electrode (6a) forms the bottom of the nanochannel (8).

Similarly, the second electrode (6b) may have a second recess (28b). The second recess (28b) may have a square or another shape. The second recess (28b) may be smaller than the first recess (28a).

The second electrode (6b) may be a ring electrode. The second electrode (6b) may have a square shape. Alternatively, the first electrode (6a), the intermediate layer (12), and the second electrode (6a) may also be designed otherwise. For example, the first electrode (6a) may be configured to have a recess, as was described above.

The respective thickness (ED1, ED2) of the first electrode (6a) and/or the second electrode (6b) (see FIG. 3) may be in a range from about 20 nm to about 50 nm. Other ranges such as a range from about 10 nm to about 100 nm or a range from about 10 nm to about 300 nm are also contemplated. The thickness may be about, at least about, or at most about 10, 20, 30, 40, 50, 60, 70, 80, 90, or 100 nm. The respective thickness (ED1, ED2) of the first electrode (6a) and/or the second electrode (6b) may be the same or different. The roughness of the surfaces of the first electrode (6a) and/or the second electrode (6b) may be less than about 0.2 nm such as about 0.2, 0.18, 0.16, 0.15, 0.14, 0.12, 0.1, 0.08, 0.06, or 0.04 nm.

The intermediate layer (12) may be made of an electrically insulating material such as aluminum oxide (Al₂O₃) or silicon dioxide (SiO₂). However, other electrically insulating materials, especially high-quality electrically insulating materials such as hafnium oxide, may also be used. The thickness (DZ) (see FIG. 3) of the intermediate layer (12) may be in the range from about 1 nm to about 5 nm, from 2.5 nm to 3.5 nm, or 1 nm to 10 nm. The thickness may be from about 1-5, 2.5-3.5, or 1-10 nm. The thickness may be about, at most about, or at lest about 1, 1.5, 2, 2.5, 3, 3.5, 4, 4.5, 5, 5.5, 6, 6.5, 7, 7.5, 8, 8.5, 9, 9.5 or 10 nm.

In the non-limiting example of FIG. 3, the first electrode (6a) and the second electrode (6b) as well as the intermediate layer (12) may have a chamfer (14) (see FIG. 3). The chamfer (14) may have a chamfer angle (α) (see FIG. 3) of about 45° in relation to the plane of the substrate (22). Alternatively, the chamfer angle (α) may be in a range named above such as from about 40° to 50°, 30° to 70°, or 0° to 85°. Furthermore, the chamfer (14) may have one or more or a plurality of areas with different chamfer angles (a) in such a way that the chamfer (14) has a bend between the different areas such as between two different areas.

The first electrode (6a) and the second electrode (6b) as well as the intermediate layer (12) are arranged in the nanochannel (8) in such a way that respective exposed surface portions (30a, 30b) of the first electrode (6a) and the second electrode (6b) are arranged adjacent to the nanochannel (12) to allow direct contact with a medium in the nanochannel (8). As a result of the chamfer (14) having a chamfer angle (α) of about 45° in relation to the plane of the substrate (22), the surface portions (30a, 30b) are increased by approximately 40% by the chamfer (14).

The first electrode (6a) and the second electrode (6b) are thus not arranged on two opposite walls of the nanochannel (8). Instead, they are separated from each other by the intermediate layer (12). The intermediate layer may have a thickness (DZ) (see FIG. 3) contributing to determining, in particular defining, the electrode distance between the first electrode (6a) and the second electrode (6b). Therefore, the electrode distance may be in the range disclosed above, for example from about 1 nm to 5 nm, 2.5 nm to 3.5 nm, or 1 nm to 10 nm. The thickness of the intermediate layer is selected to ensure the desired voltage resistance.

The nanochannel (8) may have a rectangular cross-section. Alternatively, the nanochannel (8) may also have a different cross-sectional shape such as round or oval.

The height (H) (see FIG. 3) of the nanochannel (8) may be in the range disclosed above such as from about 1 nm to 5 nm, 5 nm to 50 nm, or 3 nm to 100 nm. The width (B) (see FIG. 2) of the nanochannel (8) may be in the range disclosed above such as from 3 nm to 100 nm, 3 nm to 20 nm, or 3 nm to 10 nm.

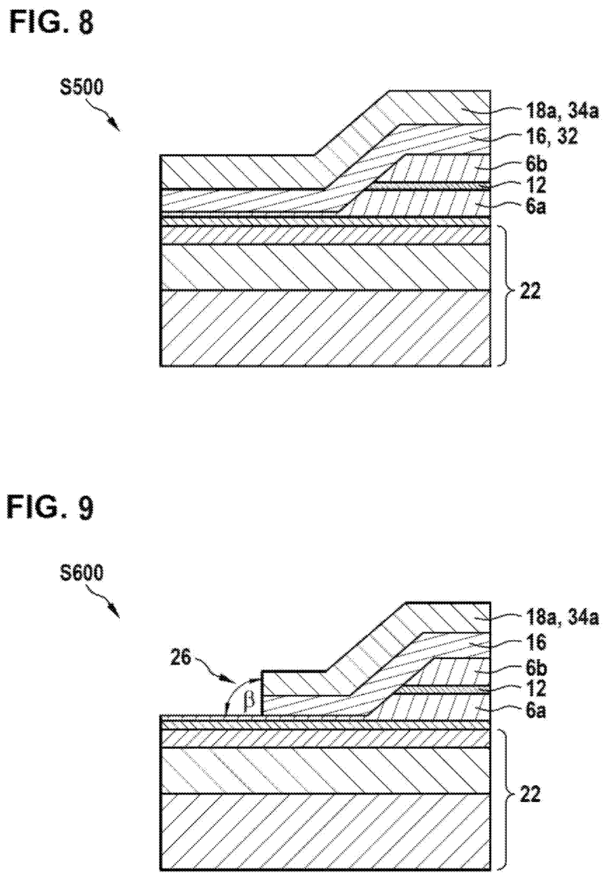

The nanochannel (8) is formed by removal of a sacrificial layer (32) (see, for example, FIG. 8), as explained in detail below. In a non-limiting example, the sacrificial layer (16) (see for example FIG. 8) includes silicon dioxide (SiO₂). Alternatively, other materials such as silicon nitride (SiN) or aluminum oxide (Al₂O₃) may also be used for the sacrificial layer.

The upper side of the nanochannel (8) may include a first coating layer (34a) made of a first coating material (18a) and a second coating layer (34b) made of a second coating material (18b) (see, for example, FIG. 11). The first coating material (18a) and/or the second coating material (18b) may include aluminum oxide (Al₂O₃). Alternatively, other materials, e.g., silicon oxide (SiO₂) may be used.

The main direction of extension of the nanochannel (8) may be arranged at an angle (Y) of about 90° (see angle (γ) in FIG. 2) to the direction of extension of the edges of the exposed surface portions (30a, 30b) of the first electrode (6a) and the second electrode (6b) (see FIG. 3).

Alternatively, the main direction of extension of the nanochannel (8) may also extend at a different angle (γ) of about 70° to 110° to a direction of extension of the edges of the exposed surface portions (30a, 30b) of the first electrode (6a) and the second electrode (6b). This does not refer to the slope of the phase of the first electrode (6a) and/or the intermediate layer material (10) and/or the second electrode (6b), which is defined by the angle (α).

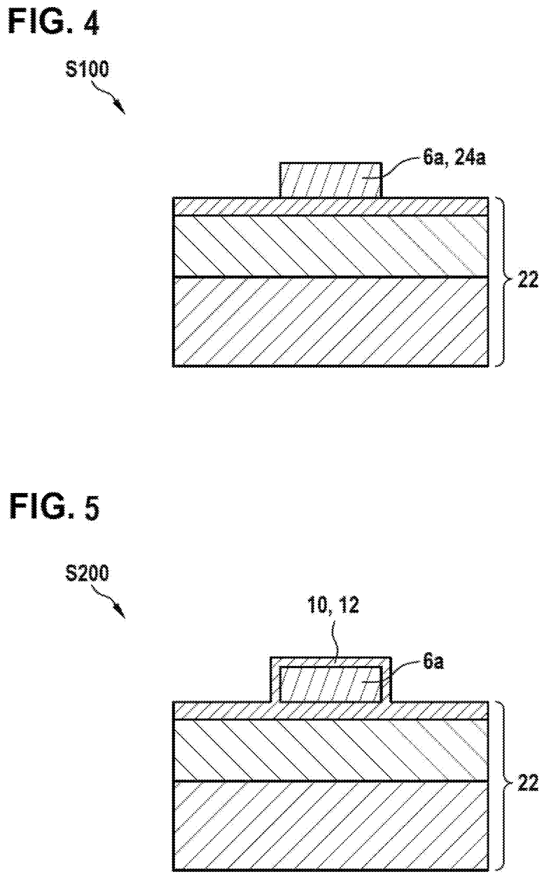

Furthermore, the nanochannel (8) may traverse the two electrodes (6a, 6b) with the intermediate layer (12) twice (see FIG. 2). The unit (4) includes the nanochannel (8) shown in FIG. 2. FIGS. 4 to 11 show sectional views of the unit (4) along the sectional line II-II.

FIGS. 4-11 illustrate a non-limiting example of a method for manufacturing of the unit (4) disclosed herein. The sequence of steps may be altered. Furthermore, several steps can also be conducted at the same time or simultaneously. Moreover, it is also possible to skip or omit one or more individual steps disclosed herein. The method steps shown may include several sub-steps, which are not shown separately.

FIG. 4 shows a first method step S100, in which the electrode material (24a), which in a non-limiting example may include platinum (Pt), is deposited on the substrate (22), in a non-limiting example on the entire surface, and subsequently structured to form the first electrode (6a). Alternatively, other materials such as titanium nitride (TiN) or other electrochemically active materials may also be used as electrode material (24a). The first electrode material (24a) may be deposited on a part of the substrate (22).

FIG. 5 shows a further method step S200, in which an intermediate layer material (10) is deposited on the entire surface of the first electrode (6a) to form the intermediate layer (12). Aluminum oxide (Al₂O₃) may be used as the intermediate layer material (10). Alternatively, other electrically insulating materials such as silicon dioxide (SiO₂) may be used. The intermediate layer material (10) may be deposited on a part of the first electrode material (24a).

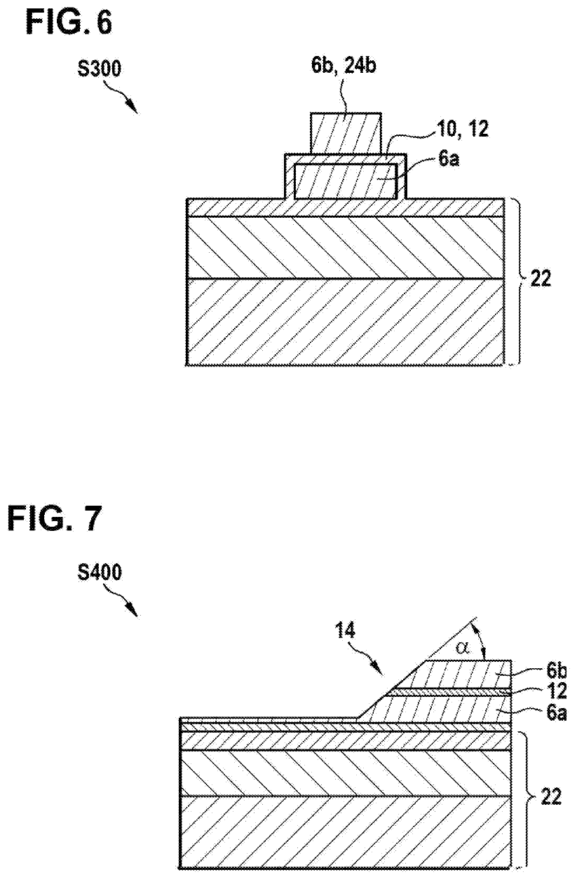

FIG. 6 shows a further method step S300, in which an additional electrode material (24b), which in a non-limiting example may be in platinum (Pt), is deposited on the intermediate layer (12). Alternatively, other materials such as titanium nitride (TiN) or other electrochemically active materials may also be used as an additional electrode material (24b). The further electrode material (24b) may be structured by lift-off, plasma etching, or ion beam etching to form the intermediate layer (12). The intermediate layer may have a first recess (28a). The second electrode (6b) may have a second recess (28b).

The first and second electrodes (6a, 6b) may be manufactured from the same electrode material (24a, 24b), making their further processing simpler. Alternatively, the two electrodes (6a, 6b) may also be manufactured from different electrode materials (24a, 24b). In a non-limiting example, the additional electrode material (24b) may be structured directly by an etching method without lift-off.

FIG. 7 shows a further method step S400, in which the first electrode (6a) and the intermediate layer (12), located between the two electrodes (6a, 6b), and the second electrode (6b) are structured in such a way that they have a chamfer (14). The chamfer (14) has a chamfer angle (α) of about 45° in relation to the plane of the substrate (22). Alternatively, the chamfer angle (α) may also be in a range disclosed above such as from about 40° to about 50°, about 30° to about 70°, about 0° to about 85°. The chamfer (14) may be formed by ion beam etching (IBE) or reactive ion etching (RIE). Alternatively, other etching methods may be used which enable anisotropic etching of the first electrode (6a), the intermediate layer (12), the second electrode (6b), or their combination, and which at the same time generally lead to the formation of the chamfer (14).

FIG. 8 shows a further method step S500, in which first a sacrificial layer material (16), is used to form the sacrificial layer (32). The first sacrificial layer material (16) may include silicon dioxide (SiO₂), or another material. The sacrificial layer material (16) may be deposited onto the first electrode (6a), the intermediate layer (12), the second electrode (6b), or a combination thereof. The sacrificial layer material (16) may be deposited onto the entire surface of the two electrodes (6a, 6b) between which the intermediate layer (12) is interposed. The sacrificial layer material (16) may be subsequently structured.

The method may subsequently include a step of using a coating material (18a) to form the first coating layer (34a). In a non-limiting example, the coating material (18a) may be aluminum oxide (Al₂O₃). The coating material (18a) may be deposited onto the sacrificial layer (32). The coating material (18a) may be deposited onto the entire surface of the sacrificial layer (32). The coating material (18a) may be structured after its deposition.

FIG. 9 shows a further method step S600, in which the sacrificial layer material (16) and the coating material (18a) are structured by ion beam etching (IBE) or reactive ion etching (RIE). More specifically, the structuring may be conducted by forming a wall (26) which is essentially vertical and has essentially 90° flank angle (B). The term “essentially” is to be understood here as being within technical manufacturing tolerances. Alternatively, the flank angle (B) can also differ slightly from the value about 90° and be in a range from about 80° to about 100°.

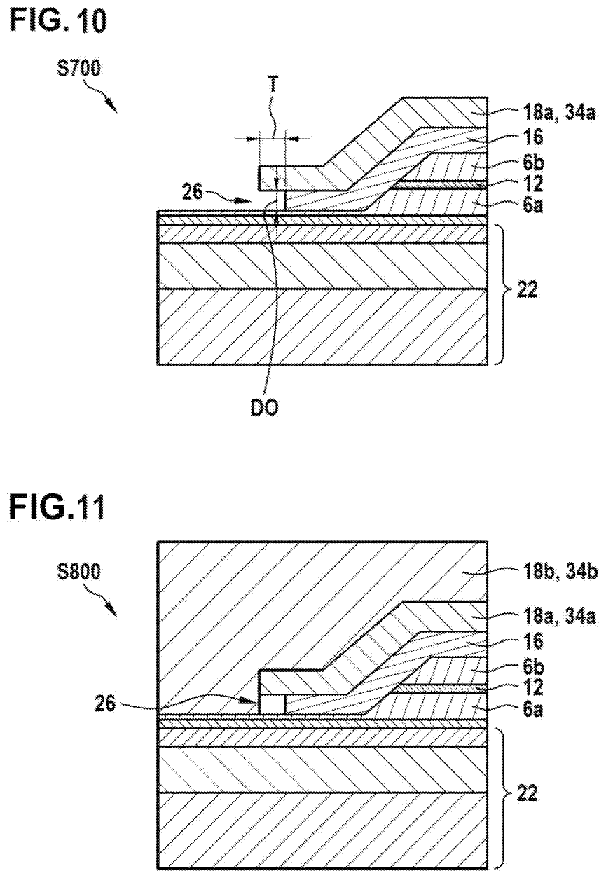

FIG. 10 shows a further method step S700, in which the sacrificial layer material (16) is partially etched under the coating material (18a) to form the nanochannel (8). The nanochannel may be partially open at this stage of the method. The partial under-etching is conducted with a ratio of approximately 1 to 1 in relation to the thickness of the sacrificial layer (DO) of the structured sacrificial layer material (16) to the lateral under-etching depth (T), running parallel to the surface of the substrate (22). For partial under-etching, aqueous tetramethylammonium hydroxide solution (TMAH) or gaseous xenon difluoride (XeF₂) may be used.

FIG. 11 shows a further method step S800, in which the additional coating material (18b) used to form the second coating layer (34b), is deposited over or on the structures/layers formed so far. The second coating layer (34b) may be thus formed above and covering the layers including the first electrode layer, the intermediate layer, the second electrode layer, the sacrificial layer, the first coating layer, or their combination. The second coating layer (34b) may be in direct contact with one or more of the structures/layers formed so far such as the first electrode layer, the second electrode layer, the sacrificial layer, the first coating layer, or their combination. In a non-limiting example, second coating layer (34b) may be deposited over the first electrode layer, the intermediate layer, the second electrode layer, the sacrificial layer, and the first coating layer while being in direct contact with only the first electrode layer (6a) and the first coating layer (34a).

In a non-limiting example, the additional coating material (18b) may be aluminum oxide (Al₂O₃). Due to the usually particularly good edge coating of the used processes (plasma CVD, sputtering, etc.) for the deposition of aluminum oxide (Al₂O₃), a closure of the nanochannel (8) is achieved by application of the second coating material (18b), especially along one side in the direction of the longitudinal extension of the nanochannel (8).

Furthermore, in a further step (not shown), a micro-channel (also not shown) may be etched for fluidic connection to the nanochannel (8).

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A method for producing a sensor unit for nucleic acid analysis having a first electrode and a second electrode in a nanochannel, the method comprising:
arranging a main direction of extension of the nanochannel at an angle γ from about 70° to about 110° to a direction of extension of edges of exposed surface portions of the first and second electrodes.

2. The method according to claim 1, wherein the angle γ is about 90°.

3. The method according to claim 1, further comprising:
depositing a first electrode material to form the first electrode in step (S100);
depositing an intermediate layer material to form an intermediate layer between the first electrode and a second electrode such that the intermediate layer defines an electrode distance between the first and second electrodes in step (S200); and
depositing a second electrode material on the intermediate layer to form the second electrode in step (S300).

4. The method according to claim 3, further comprising structuring the first electrode material of the first electrode, an intermediate layer material of the intermediate layer, the second electrode material of the second electrode, or their combination to form a chamfer with at least one chamfer angle α in step (S400).

5. The method according to claim 3, further comprising:
depositing a sacrificial layer material on the first electrode, the intermediate layer, the second electrode, or their combination to form a sacrificial layer, and depositing a coating material on the sacrificial layer to form a coating layer in step (S500);
structuring the sacrificial layer and/or the coating material in step (S600); and
partially under-etching the deposited and structured sacrificial layer to form the nano-channel in step (S700).

6. The method according to claim 5, further comprising depositing an additional coating material on the deposited coating material to form a second coating layer to close the nanochannel in step (S800).

7. A nucleic acid analysis sensor unit comprising:
a first and second electrodes located in a nanochannel, the first and second electrodes each having exposed surface portions, the nanochannel having a main direction of extension arranged at an angle γ from about 70° to about 110° to a direction of extension of edges of the exposed surface portions.

8. The sensor unit according to claim 7, wherein the angle γ is about 90°.

9. The sensor unit according to claim 7, further comprising an intermediate layer material forming at least one intermediate layer between the first and second electrodes, the intermediate layer defining an electrode distance between the first and second electrodes.

10. The sensor unit according to claim 9, wherein the intermediate layer has a thickness of about 1 nm to about 10 nm.

11. The sensor unit according to claim 9, wherein the electrode distance is about 1 nm to about 5 nm.

12. The sensor unit according to claim 9, wherein the first electrode, the intermediate layer, the second electrode, or a combination thereof include a chamfer with at least one chamfer angle α.

13. The sensor unit according to claim 12, wherein the at least one chamfer angle α is in a range from about 0° to about 85°.

14. The sensor unit according to claim 13, wherein the at least one chamfer angle α is in a range from about 30° to about 70°.

15. The sensor unit according to claim 9, further comprising a structured sacrificial layer material on the first electrode, the intermediate layer, the second electrode, or a combination thereof; and
a first coating layer configured on the structured sacrificial layer.

16. The sensor unit according to claim 15, further comprising a second coating material on the first coating material, the second coating material closing the nanochannel.

17. A DNA or RNA sequencing analysis system comprising:
first and second containers connected by a nanochannel;
a nucleic acid analysis sensor unit including a first and second electrodes, located in the nanochannel, adjacent each other and each including one or more exposed surface portions with one or more edges,
the nanochannel having a main direction of extension arranged at an angle γ from about 70° to about 110° to a direction of extension of the one or more edges.

18. The analysis system of claim 17, further comprising an intermediate layer located between and separating the first and second electrodes, the separation measuring about 1 nm to about 5 nm.

19. The analysis system of claim 18, wherein the first electrode, the intermediate layer, the second electrode, or a combination thereof include a chamfer with at least one chamfer angle α of about 0° to about 85°.

20. The analysis system of claim 19, further comprising a structured sacrificial layer material on the first electrode, the intermediate layer, the second electrode, or a combination thereof; and a first coating layer configured on the structured sacrificial layer.
